(19) United States
(12) Patent Application Publication
(10) Pub. No.: US 2024/0260739 A1
(43) Pub. Date: Aug. 8, 2024

(54) Surgical cleaning device with adapter

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/425,617

(22) Filed: Jan. 29, 2024

(30) Foreign Application Priority Data
Feb. 3, 2023 (EP) 23154817.3

(51) Int. Cl. A46B 15/00; A46B 9/02; A46B 11/00; A46B 13/06; A61B 90/70

(52) U.S. Cl. CPC A46B 15/0053; A46B 9/028; A46B 11/0006; A46B 11/0072; A46B 13/06; A61B 90/70; A46B 2200/3073

(57) ABSTRACT

A device for the surgical cleaning of infected implants, the device containing a brush head containing a bristle region having a plurality of bristles; a supply element configured to transport a cleaning liquid to the brush head; a discharge element configured to remove liquid from the brush head; and a connecting element configured to releasably connect the supply element and the discharge element to an external lavage device.

### CROSS-REFERENCE TO RELATED APPLICATIONS

This application claims priority pursuant to 35 U.S.C. 119(a) to European application Ser. No. 23/154,817.3, filed Feb. 3, 2023, which application is incorporated herein by reference in its entirety.

### FIELD OF THE INVENTION

The present invention relates to the field of medical technology, in particular devices and methods for use in the surgical cleaning of infected tissue.

The subject matter of the invention is in particular a surgical cleaning device which can be usable as a single-use device for the mechanical cleaning and irrigation of infected joint endoprostheses within the scope of DAIR procedures. The cleaning device is suitable for the manual brushing of surfaces of joint endoprostheses; the mechanically detached contaminants can be removed by at the same time by cleaning liquid jets exiting the cleaning device. The cleaning liquid can be connected to a conventional jet lavage system, whereby a cleaning liquid can be pumped into the cleaning device. The cleaning liquid can preferably exit the cleaning device in a pulsed manner.

### TECHNICAL BACKGROUND

In septic bone surgery, the cleaning of debrided bone and soft tissue areas in the revision of infected joint endoprostheses and the cleaning of infected osteosynthesis plates by means of lavage is known. Medical irrigation devices, so-called lavage systems, are used for this purpose. These lavage systems are also referred to herein as “lavage devices.” Known cleaning liquids are physiological saline solution, Ringer's solution, and Ringer's lactate solution. The aim of lavage is to remove residues of the infected tissue, biofilm residues, blood, and wound secretions. Lavage systems have long been known. Devices according to the patent specifications EP3187208, U.S. Pat. No. 4,583,531, U.S. Pat. No. 5,779,702, U.S. Pat. No. 6,059,754 and U.S. Pat. No. 20,191,51531 are examples thereof. However, a problem is that firmly adhering biofilms and particularly biofilms in undercut regions can be detached only conditionally or even not at all by means of the conventional jet lavage systems.

In septic surgery, the so-called DAIR procedures (debridement, antibiotics and implant retention) for treating infected knee joint endoprostheses, hip joint endoprostheses and shoulder joint endoprostheses are on the rise. The aim of this surgical technique is to fight the infection of the joint endoprosthesis and of the surrounding tissue, after opening the joint, by debridement and subsequent local application of antibiotics such that the artificial joint is preserved. The implantation of a revision joint endoprosthesis, which is cost-intensive and puts the patient under strain, is to be avoided.

These procedures are used in particular in the case of artificial joint endoprostheses that are infected at an early stage. Movable implant components, such as PE inlays, are first removed from the artificial joint. Infected tissue is debrided and an attempt is made to remove biofilms, which adhere to the metal surfaces of the joint endoprosthesis, as completely as possible by means of brushing and lavage. New movable joint components, such as PE Inlays, are then inserted into the cleaned joint endoprosthesis. The removal of the biofilms on the metal surfaces, in particular on the condyles of knee joints, is problematic here. Biofilms can be extremely sticky and adherent. This means that, despite brushing and subsequent lavage, biofilms or biofilm residues can remain on the metal surfaces. These biofilm residues contain living microorganisms and can lead to a reinfection of the joint endoprosthesis and the surrounding tissue.

### PREFERRED EMBODIMENTS

The object of the present invention is to solve one or more of the above-described and other problems of the prior art. An object of the invention is to detach biofilms by brushing the implant surface and simultaneously to remove the detached biofilm residues from the implant surface using a cleaning liquid. As a result, after being mechanically detached, the sticky biofilms cannot adhere to the implant surface again. A further object of the invention is to immediately remove the biofilm residues mixed with the cleaning liquid from the joint site, so that a deposition of the detached biofilm residues on the surrounding tissue, the joint capsule, can be effectively prevented.

The device can preferably allow manual brushing, also in undercuts, and at the same time the contaminants, such as biofilm residues, which are detached by brushing can be immediately removed by jets of irrigation liquid, so that the contaminants cannot attach to the cleaned surfaces. The device itself can preferably be without an active drive device of its own and also without a pump. The device can preferably be manufactured as simply and cost-effectively as possible from plastic parts. Furthermore, the device can preferably be sterilized by ethylene oxide or gamma sterilization.

Some or all objects are achieved by methods and devices described herein, in particular those described in the claims.

Preferred embodiments of the invention are described below.

A first embodiment describes a device for the surgical cleaning of infected implants, containing a brush head containing a bristle region having a plurality of bristles; a supply element configured to transport a cleaning liquid to the brush head; a discharge element configured to remove liquid from the brush head; and a connecting element configured to releasably connect the supply element and the discharge element to an external lavage device.

A second embodiment describes a device according to the first embodiment, wherein the device is configured to achieve a ratio of the delivery rate of the discharge element to the delivery rate of the supply element of at least 1.

A third embodiment describes a device according to the first or second embodiment, wherein the brush head is configured to exert an oscillating force on the bristles.

A fourth embodiment describes a device according to any one of the preceding embodiments, wherein the device comprises a drive element for generating the oscillating force, the drive element preferably being configured for driving by means of the flow of the cleaning liquid through the supply element.

A fifth embodiment describes a device according to any one of the preceding embodiments, further comprising a valve, which is arranged between the connecting element and the brush head and is configured to control the flow of a cleaning liquid through the supply element.

A sixth embodiment describes a device according to any one of the preceding embodiments, wherein the brush head comprises bristles of different lengths in order to facilitate the cleaning of undercuts, the bristles preferably being arranged arcuately in the bristle region.

A seventh embodiment describes a device according to any one of the preceding embodiments, wherein the supply element comprises an outlet opening arranged within the bristle region.

An eighth embodiment describes a device according to any one of the preceding embodiments, wherein the discharge element has a suction opening arranged at the proximal end of the bristle region.

A ninth embodiment describes a device according to any one of the preceding embodiments, wherein the device is configured to guide cleaning liquid from the supply element through the bristles and to release said cleaning liquid at the tip of the bristles.

A tenth embodiment describes a device according to any one of the preceding embodiments, wherein the device does not comprise a pump.

An eleventh embodiment describes a device according to any one of the preceding embodiments, wherein the supply element, and preferably also the discharge element, are guided through the connecting element.

A twelfth embodiment describes a device according to any one of the preceding embodiments, further comprising a handle element arranged between the connecting element and the bristle element, the handle element being configured for a user to manually guide the device.

A thirteenth embodiment describes a device according to the twelfth embodiment, further comprising a stem element which connects the handle element to the brush head.

A fourteenth embodiment describes a device according to the thirteenth embodiment, wherein the stem element has a length of 5 to 10 cm and/or has a diameter of at most 10 mm.

A fifteenth embodiment describes a device according to any one of the preceding embodiments, wherein the supply element comprises a flexible tube and/or has a length of at least 80 cm.

### DETAILED DESCRIPTION

With respect to the embodiments described herein, the elements of which “contain,” or “comprise,” a particular feature (for example, a material), in principle a further embodiment is always contemplated in which the relevant element consists solely of the feature, i.e., does not comprise any other constituents. The word “comprise” or “comprising” is used herein synonymously with the word “contain” or “containing.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa. All such considered combinations are not explicitly listed in all instances, simply in order to keep the description brief. Technical solutions known to be equivalent to the features described herein are also intended in principle to be encompassed by the scope of the invention.

A first aspect of the invention relates to a device for the surgical cleaning of infected implants, the device containing a brush head containing a bristle region having a plurality of bristles; a supply element configured to transport a cleaning liquid to the brush head; a discharge element configured to remove liquid from the brush head; and a connecting element configured to releasably connect the supply element and the discharge element to an external lavage device.

The device is provided in particular for cleaning bacterially infected tissue, for example during a surgical procedure. The device can preferably be used for cleaning infected implants.

The device comprises a brush head on which a plurality of bristles is arranged in a bristle region. The bristles are preferably formed from a biocompatible polymer, for example polyethylene, polypropylene, polyamide, or the like.

The device comprises a supply element which is configured to transport a cleaning liquid. In particular, a cleaning liquid can be guided from a connecting element to the brush head by means of the supply element in order to be released in the region of the brush head.

The device comprises a discharge element which is designed to receive and transport liquid. The discharge element can be used to remove cleaning liquid in the used or contaminated state, said cleaning liquid having been initially released at the brush head via the supply element. Infected tissue and biofilms can thereby be removed from a patient, so that there is no risk of pathogens remaining or even being distributed in the patient. In particular, a re-adhering of bacteria from biofilms to an implant can be avoided.

The devices described herein are particularly suitable for operation with an external lavage device. Examples of lavage devices with which the devices described herein can be operated are described, for example, in EP2662146A2, U.S. Pat. No. 4,583,531A, U.S. Pat. No. 4,278,078A, and U.S. Pat. No. 5,542,918A. Such lavage devices can comprise a pump, which can be equipped, for example, with an electric motor or a compressed air motor. The devices described herein can preferably be operated with lavage devices which can generate pulsed bursts of spray. The lavage devices can, for example, comprise pumps such as are described in EP2619415A1, EP2910270A1 or EP2873856A1.

The device described herein is equipped with a connecting element for connection to such a lavage device. The connecting element can be connectible to an external lavage device in a form-closed or force-closed connection. For example, the connecting element can comprises a latching element in order to connect the device to a lavage device. The connecting element can in particular be configured to connect the supply element and the discharge element in a liquid-conducting manner to a lavage device. The connecting element can in particular comprise an opening configured to dispense liquid from the discharge element to a lavage device. The connecting element can furthermore comprise an opening configured to receive a cleaning liquid from a lavage device in order to guide the cleaning liquid into the supply element.

For this reason, the devices described herein do not themselves require a pump. Accordingly, the devices described herein preferably do not have their own pump. A pump is a device which can bring about the transport of a liquid by actively changing the pressure of the liquid to be transported.

In some embodiments, the device is configured to achieve a ratio of the delivery rate of the discharge element to the delivery rate of the supply element of at least 1. This means that the device is configured to simultaneously discharge at least as much liquid via the discharge element as is released at the brush head via the supply element. It can thereby be better ensured that loosened biofilms and detached infected tissue can be effectively removed from the patient.

In some embodiments, the ratio of the delivery rate of the discharge element to the delivery rate of the supply element is at least 1.0; 1.2; 1.5; or at least 2. This can be achieved, for example, in that the inner diameter of the discharge element is dimensioned to be correspondingly greater compared to the inner diameter of the supply element.

In some embodiments, the brush head is configured to exert an oscillating force on the bristles. For example, the brush head can be movable, for example due to elastic material properties of the device or due to the incorporation of joints. The bristles can thereby be set in vibration. Better detaching of infected tissue or of biofilms can thereby be achieved.

In some embodiments, the device comprises a drive element for generating such an oscillating force. The drive element can be an active drive element, for example an electric motor, or a passive drive element can be used. An example of a passive drive element is a vibration motor which is operated by the flow of a cleaning liquid through the supply element.

For example, a blade wheel which is driven by a liquid flow and which has a non-symmetrical mass distribution can be used for this purpose.

Further examples of drive elements are ball vibration motors and roller vibration motors. A circular movement of a spherical mass body can create an imbalance which leads to vibrations of the stem element and/or of the brush head, as a result of which the cleaning effect can be intensified.

The oscillating force can also be generated by bursts of spray of a cleaning liquid. For this purpose, the device can be connected to a lavage device which allows the pulsed dispensing of a cleaning liquid. For this purpose, the external lavage device can be equipped with a pump and a controller which enables such pulsed operation.

In some embodiments, the device comprises a valve configured to control the flow of a cleaning liquid through the supply element. The valve can preferably be arranged between the connecting element and the brush head.

The brush head can comprise bristles of different lengths in order to facilitate the cleaning of undercuts. For example, the bristles of different lengths can be arranged arcuately on the brush head, as shown, for example, in FIG. 3. For example, the bristles in the distal region of the brush head can be longer than the bristles at the proximal region of the brush head. The distal region of the brush head is the side which is arranged furthest away from the connecting element. The proximal region of the brush head is the side which points in the direction of the handle element (if present) and the connecting element.

In some embodiments, the supply element comprises an outlet opening arranged within the bristle region. The device preferably comprises a plurality of outlet openings. The outlet openings can be arranged on the brush head in the vicinity of the bristles, preferably within the bristle region, or on the bristles themselves, or both. The bristles can be hollow in order to allow the transport of a cleaning liquid. The device can be configured to guide cleaning liquid from the supply element through the bristles and to release said cleaning liquid at the tip of the bristles.

The supply element and/or the discharge element is preferably guided through the connecting element. They preferably run within the connecting element and are thereby better protected against damage or disturbance.

The device can comprise a handle element configured for a user to manually guide the device. The handle element can be arranged between the connecting element and the brush head. Using the handle element, a user can securely grasp and hold the brush head, and can manually position and guide the brush head in a suitable manner during use.

The device can furthermore comprise a stem element which connects the handle element to the brush head. Accordingly, the stem element is arranged between the handle element and the brush head. The stem element can preferably be flexible in order to enable a movement of the brush head, as described in more detail above. The stem element can, for example, have a length of 5 to 10 cm. The stem element can have a diameter of 10 mm or less, for example less than 9 mm or less than 7 mm. This can make it easier for the user to better reach body regions that are difficult to access, for example in the case of DAIR procedures on infected knee joint endoprostheses. The stem element preferably has a greater outer diameter than the discharge element.

Preferably, the stem element and the discharge element are arranged parallel to one another.

Preferably, the stem element and the discharge element are directly connected to one another. The stem element and the discharge element are preferably flexible. In some embodiments, the device is made partially or completely of plastic. In particular, the brush head, stem element, handle element, and/or connecting element can each comprise a plastic, or consist completely of plastic.

In some embodiments, the device is configured for sterilization using ethylene oxide or gamma sterilization, i.e., it can be sterilized by the methods mentioned without functional impairment.

The supply element can comprise a flexible tube. The supply element can have a length of at least 80 cm. In some embodiments, the length of the supply element in the region between the connecting element and the handle element or the brush head is at least 80 cm. This affords the user better movability of the device without the need to move the external lavage device at the same time.

In some embodiments, the brush head comprises an angled outflow channel. In relation to the orientation of the bristles, the outflow channel can be arranged at an angle of more than 15°, for example approximately 45°. Bursts of spray of the cleaning liquid can thereby be released into the region next to the brush head in order to improve the cleaning performance of the brush. The cleaning liquid can be released at the outer end of the outflow channel via an outflow opening.

A further aspect relates to a therapy method in which a device described herein is used to remove biofilms or infected tissue. In this case, biofilms or infected tissue is removed by means of the bristles and by means of a cleaning liquid released via the supply element, and the cleaning liquid together with the material to be removed is taken out of the patient via the discharge element. The device can be connected to an external lavage device. In one embodiment, pulsating bursts of spray of the cleaning liquid are released via the brush head. In one embodiment, an oscillating force is applied via the bristles, as described herein.

The cleaning liquid can comprise a detergent, a buffer substance, and/or an antibacterial active substance. The cleaning liquid can comprise a physiological saline solution, Ringer's solution or Ringer's lactate solution.

The device can be used for a debridement and in particular for DAIR procedures. The device can be used, for example, in the context of a knee or hip joint operation, in particular in the case of revision surgery.

### EXAMPLES

The invention is further illustrated below using examples which are, however, not to be understood as limiting. It will be apparent to a person skilled in the art that other equivalent means may be used similarly in place of the features described here.

### FIGURES

FIG. 1 shows by way of example an embodiment of a device 100 according to the invention. The device comprises a brush head 101. The brush head 101 comprises a stem element 112 with a bristle region 102. A plurality of bristles 103 is arranged in tufts in the bristle region 102. The stem element 112 comprises a suction opening 109 adjacent to the bristle region 102. The suction opening 109 can serve to receive and remove cleaning liquid which, by being used with the device according to the invention, detached infected tissue or biofilms. The brush head 101 is releasably connected to a handle element 111. The handle element 111 further comprises a valve for controlling a supply element 104. The supply element 104 is guided within the handle element 111 and the brush head 101. The supply element 104 serves to supply a cleaning liquid into the brush head 101. Furthermore, the device comprises a discharge element 105 which can serve to discharge the cleaning liquid received by the suction opening 109. The supply element 104 and the discharge element 105 are each guided to a connecting element 106 which is provided for connection to an external lavage device. For this purpose, the connecting element 106 comprises a latching element 125 which can be connected to a lavage device.

FIG. 2 shows the connecting of a device 100 according to the invention to an external lavage device 200. In this process, the connecting element 106 of the device 100 is connected to a lavage device 200 in a form-closed connection, the supply element 104 and the discharge element 105 being connected in a liquid-tight manner to corresponding connections of the lavage device 200.

FIG. 3 shows a cross-sectional view of a portion of the brush head of a device according to the invention. The supply element 104 is configured to guide cleaning liquid to the bristle region 102. In the example shown here, the bristle region 102 comprises hollow bristles 103 which are connected to the supply element 104 in a liquid-conducting manner. Cleaning liquid can thus be guided through the hollow bristles 103 to an outlet opening 108 at the tip 110 of the bristles, where the cleaning liquid can be released out of the outlet opening 108. The cleaning liquid released out of the outlet openings is shown symbolically here in the form of drops. In this example, the bristles 103 are of different lengths. The bristles 103 have a greater length at the distal end of the brush head (shown on the left in FIG. 3) than at the proximal end of the brush head (shown on the right in FIG. 3), so that an overall arcuate arrangement results with respect to the tips of the bristles. Such a geometry can be advantageous for removing biofilms and bacterially loaded tissue in the zone of body regions that are difficult to access, in particular in the region of undercuts. An example of this are DAIR procedures on infected knee joint endoprostheses, wherein cleaning is also required in particular in the gap between the femoral component and the tibial component of the joint.

FIG. 4 shows a plan view of the portion of a brush head 101 shown in FIG. 3. A plurality of bristles 103 is arranged within the bristle region 102, the bristles each comprising a cavity which can guide a cleaning liquid from the supply element 104 to an outlet opening 108 at the tip of the bristles.

FIG. 5a shows a cross-sectional view of an embodiment of a device 100 according to the invention which is equipped with a valve 107. The valve is set up to control the flow of a cleaning liquid through the supply element 104. FIG. 5a shows the valve 107 in an open configuration, which allows a free flow of a cleaning liquid from an external lavage device 200 through the supply element 104 to the brush head 101. The cleaning liquid can thus be released in the region of the brush head 101.

FIG. 5b shows the device shown in FIG. 5a with a valve 107 in a closed configuration. The cleaning liquid, which is pumped out of the lavage device 200 into the device 100, can be conducted via the supply element 104 only up to the closed valve 107 and cannot reach into the region of the brush head 101. Therefore, when the valve 107 is closed, the cleaning liquid is not released at the brush head 101.

FIG. 6 shows a cross-sectional view of a brush head 101 of a device according to the invention. In this example, all bristles have substantially the same length. A suction opening 109 is arranged adjacent to the bristle region 102. The suction opening allows used cleaning liquid, and contaminants that are to be removed, to be received and discharged via the discharge element 105. The flow direction of the liquid to be transported is indicated by arrows.

FIG. 7 shows a cross-sectional view of a device 100 according to the invention. The flow direction of the liquid to be transported is indicated by arrows. Said liquid passes through the discharge element 105 from the suction opening 109 toward the connecting element 106, where said liquid can be received by an external lavage device.

FIG. 8 shows an embodiment of a device according to the invention with a removable brush head 101. The brush head 101 can be detachably connected to the handle element 111 of the device by being fitted onto a grommet 114. The supply element 104 and the discharge element 105 are guided through the grommet 114, and protrude, at the end thereof, beyond the grommet in order to allow a form-closed connection to the brush head 101.

FIG. 9 shows an isometric cross-sectional view of a handle element 111. In this example, the handle element is formed from two half shells which are connected to one another via latching elements 115. The supply element 104 and the discharge element 105 are guided through the interior of the handle element 111 and through a grommet 114 at the housing opening of the handle element.

FIG. 10 shows an embodiment of a device according to the invention which is equipped with a drive element 120. The drive element 120 is configured to generate an oscillating force which can be transmitted to the brush head 101 and in particular the bristles 103 in order to increase the cleaning performance of the device. The drive element 120 and a valve 107 are arranged on the handle element 111. The device can be connected to an external lavage device by means of a connecting element 106, with the supply element 104 and the discharge element 105 being connected to the lavage device in a liquid-conducting manner.

FIG. 11 shows a plan view of a cross section through a drive element 120. In this example, the drive element is a passive drive element which does not require its own power supply, but can instead be driven by the cleaning liquid flowing through. For this purpose, the drive element 120 is connected to the supply element 104 in a liquid-conducting manner, so that the cleaning liquid is conducted through an inflow opening 121 into the drive element 120, flows through a channel within the drive element, thereby moving a ball 123 on a circular path, and passes through an outflow opening 122 into the supply element 104 again. The flow direction of the cleaning liquid is indicated by arrows. This functional principle is used in a similar manner in pneumatic ball vibration motors.

FIG. 12 shows a side view of a cross section through a drive element 120. A cleaning liquid flows through the supply element 104 from an external source into the inflow opening 121 of the drive element 120, and flows via the outflow opening 122 further toward the brush head of the device. The two half shells of the handle element 111 are held together by latching elements 115.

FIG. 13a shows a cross-sectional view of a brush head 101 which has an angled outflow channel 124. In relation to the orientation of the bristles, the outflow channel 124 is arranged at an angle of more than 15°, in this example approximately 45°. Bursts of spray of the cleaning liquid can thereby be released into the region next to the brush head in order to improve the cleaning performance of the brush. The cleaning liquid can be released at the outer end of the outflow channel 124 via an outflow opening 122.

FIG. 13b shows a plan view of the brush head shown in FIG. 13a. The outflow openings are arranged along a side edge of the brush head in the bristle region 102.

FIG. 14a shows a cross-sectional view of a brush head 101 which has an arranged outflow channel 124 connecting the supply element 104 in a liquid-conducting manner to bristles 103, which comprise a liquid-conducting cavity, so that a cleaning liquid can be released to an outflow opening 122 at the tip 110 of the bristles 103.

FIG. 14b shows a plan view of the brush head shown in FIG. 14a. The variants shown in FIG. 13 and in FIG. 14 can be used alternatively or in combination in the device according to the invention.

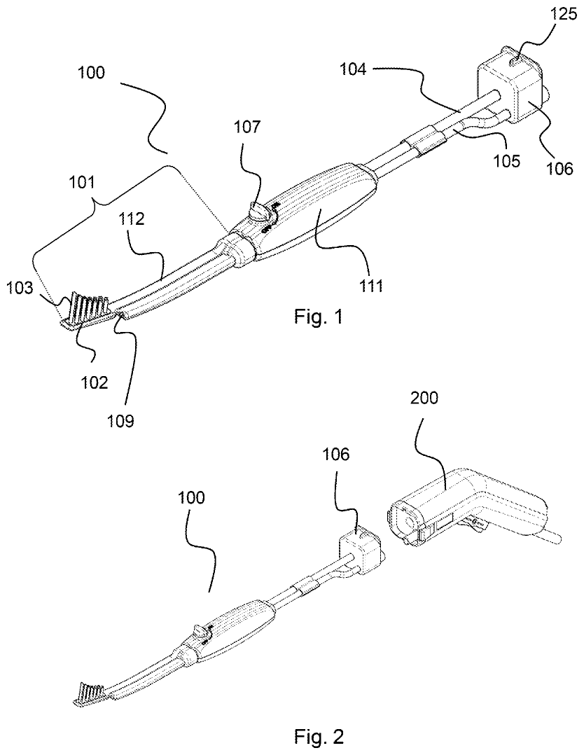

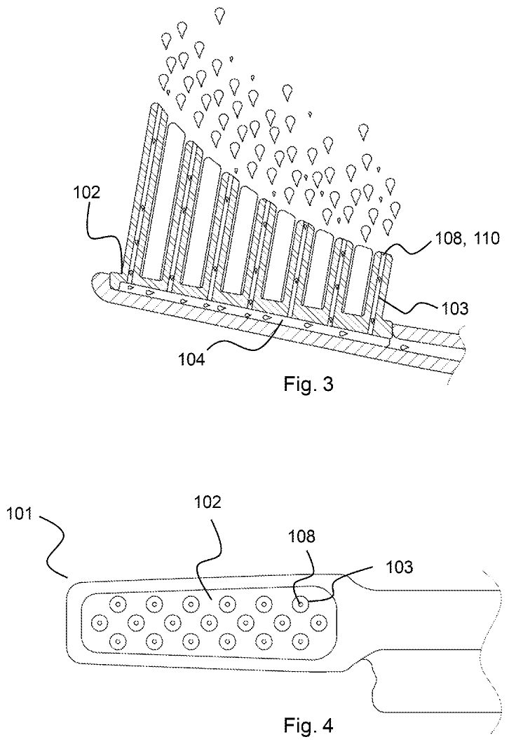

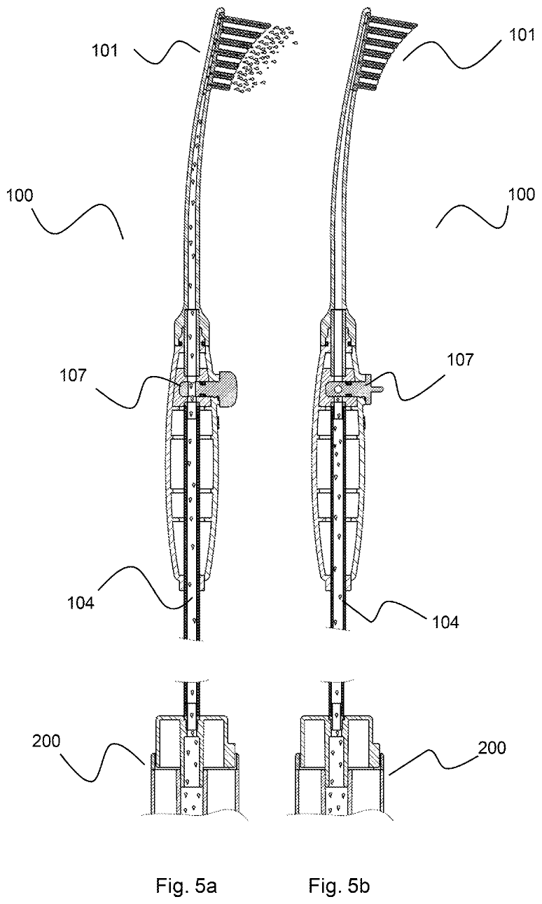

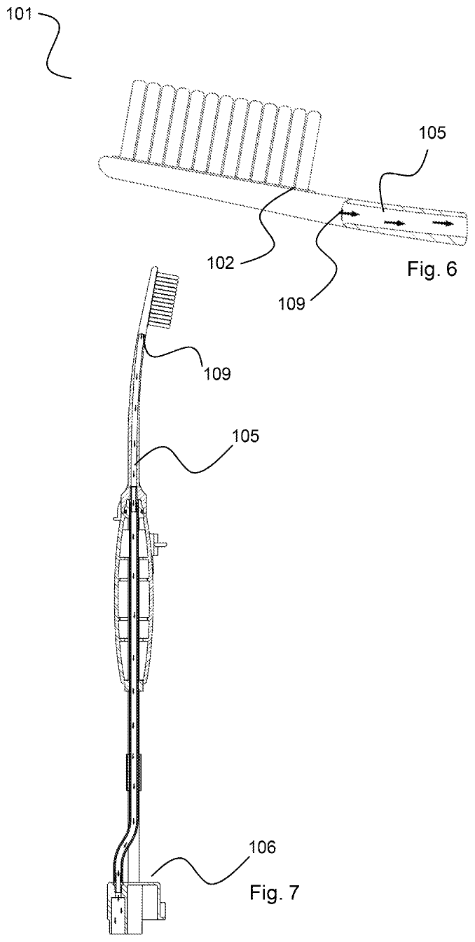

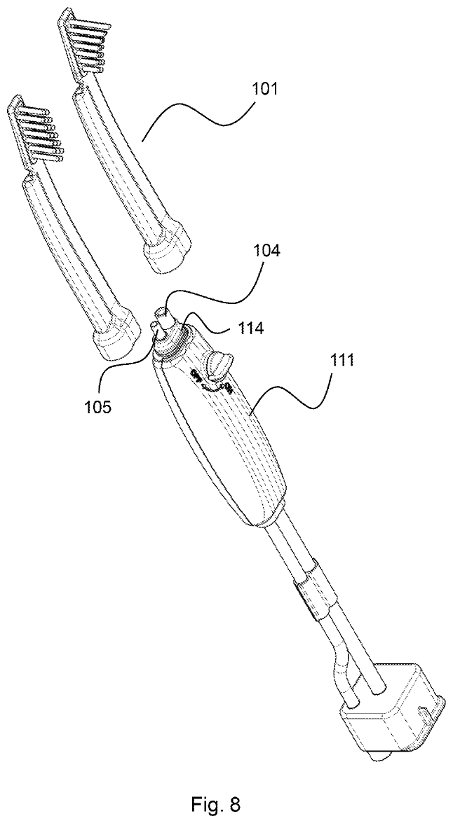

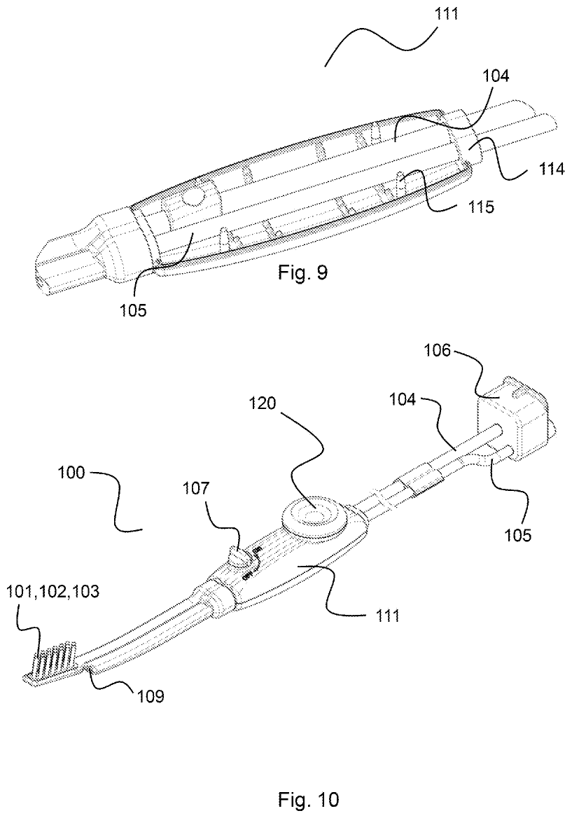

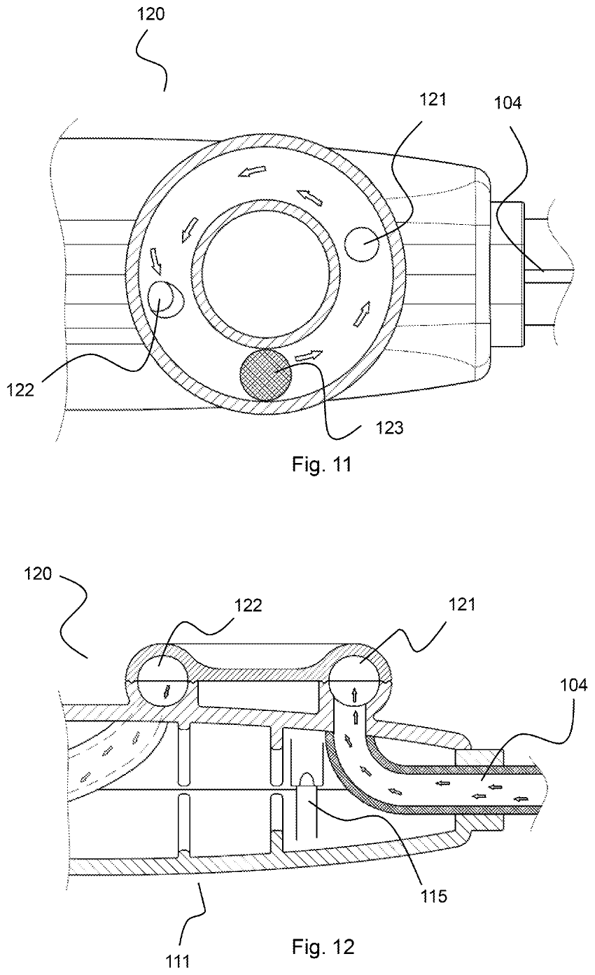

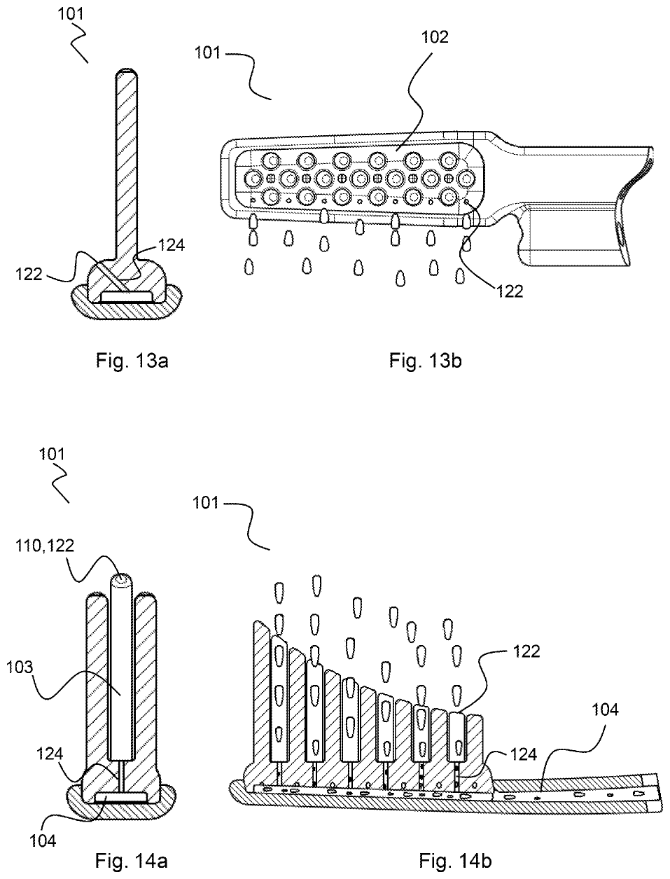

### APPLICATION EXAMPLE 1

During a surgical procedure, the device can, for example, be used as follows for debridement, wherein the following steps a) to l) are carried out in the order indicated:
a) connecting a lavage device to a cleaning liquid reservoir,
b) connecting the adapter of the device to the lavage device,
c) starting up the motor of the lavage device,
d) pumping the cleaning liquid by means of the lavage device through the supply element to the brush head of the device,
e) opening the valve in the handle element,
f) as a result, introducing the irrigation liquid along the stem element into the brush head,
g) manually brushing the surface to be cleaned, while at the same time the cleaning liquid leaves the brush head or the hollow bristles in the form of spray jets,
h) rinsing the detached contaminants away from the surface to be cleaned, by means of the cleaning liquid,
i) suctioning the used cleaning liquid from step h) through the suction element and through the connecting element into the lavage device,
j) switching off the pumping function of the lavage device,
k) separating the connecting element from the lavage device, and
l) disposing of the device.

### LIST OF REFERENCE SIGNS

100 Device
101 Brush head
102 Bristle region
103 Bristle
104 Supply element
105 Discharge element
106 Connecting element
107 Valve
108 Outlet opening
109 Suction opening
110 Tip of the bristle
111 Handle element
112 Stem element
114 Grommet
115 Latching element (on the handle element)
120 Drive element
121 Inflow opening
122 Outflow opening
123 Ball
124 Outflow channel
125 Latching element (on the connecting element)
200 Lavage device

1. A device for the surgical cleaning of infected implants, containing:
a brush head containing a bristle region having a plurality of bristles;
a supply element configured to transport a cleaning liquid to the brush head;
a discharge element configured to remove liquid from the brush head; and,
a connecting element configured to releasably connect the supply element and the discharge element to an external lavage device.

2. The device according to claim 1, wherein the device is configured to achieve a ratio of the delivery rate of the discharge element to the delivery rate of the supply element of at least 1.

3. The device according to claim 1, wherein the brush head is configured to exert an oscillating force on the bristles.

4. The device according to claim 1, wherein the device comprises a drive element for generating the oscillating force, wherein the drive element is preferably configured for driving by means of the flow of the cleaning liquid through the supply element.

5. The device according to claim 1, further comprising a valve, which is arranged between the connecting element and the brush head and is configured to control the flow of a cleaning liquid through the supply element.

6. The device according to claim 1, wherein the brush head comprises bristles of different lengths in order to facilitate the cleaning of undercuts, wherein the bristles are preferably arranged arcuately in the bristle region.

7. The device according to claim 1, wherein the supply element comprises an outlet opening arranged within the bristle region.

8. The device according to claim 1, wherein the discharge element comprises a suction opening arranged at the proximal end of the bristle region.

9. The device according to claim 1, wherein the device is configured to conduct cleaning liquid from the supply element through the bristles and to release said cleaning liquid at the tip of the bristles.

10. The device according to claim 1, wherein the device does not comprise a pump.

11. The device according to claim 1, wherein the supply element, and preferably also the discharge element, are guided through the connecting element.

12. The device according to claim 1, further comprising a handle element which is arranged between the connecting element and the brush head, wherein the handle element is configured for a user to manually guide the device.

13. The device according to claim 12, further comprising a stem element which connects the handle element to the brush head.

14. The device according to claim 13, wherein the stem element comprises a length of 5 to 10 cm and/or comprises a diameter of at most 10 mm.

15. The device according to claim 1, wherein the supply element comprises a flexible tube and/or comprises a length of at least 80 cm.
